(19) United States
(12) Patent Application Publication
(10) Pub. No.: US 2007/0048288 A1
(43) Pub. Date: Mar. 1, 2007

(54) Shear thinning polymer cell delivery compositions

(76) Inventors: SuPing Lyu, Maple Grove, MN (US); Brian Fernandes, Roseville, MN (US); Matthew A. Bergan, Forest Lake, MN (US)

Correspondence Address: MUETING, RAASCH & GEBHARDT, P.A., P.O. BOX 581415, MINNEAPOLIS, MN, 55458, US

(21) Appl. No.: 11/215,670

(22) Filed: Aug. 30, 2005

(51) Int. Cl. A61K 35/12; A61K 31/74; A61K 38/18; A61K 31/727; A61K 31/765

(52) U.S. Cl. 424/93.7; 424/78.38; 424/85.1; 514/54; 514/12; 514/573; 514/56

(57) ABSTRACT

Cell delivery compositions including shear thinning polymers and their use in cell delivery are described. The cell delivery compositions include shear thinning polymers that confer higher viscosity when at rest and decreased viscosity when subject to shear stress when dissolved or suspended in a carrier liquid. These shear thinning properties can facilitate cell delivery. Shear thinning polymer solutions may be used to deliver cells to particular tissue sites in a subject.

### BACKGROUND

Cell therapy is a relatively new method for repairing diseased or damaged organs. For patients with a variety of conditions, cell-based therapies represent a potential cure. Cell therapy can be roughly divided into two principally different approaches: (1) direct implantation of cells; and (2) implantation of engineered constructs such as scaffolds. Direct implantation involves delivering the cells directly to a particular location within a body. Implantation of engineered constructs, on the other hand, introduces cells within an engineered device or material that remodels itself in vivo.

Cell therapy can be used to repair a wide variety of tissues and organs. However, while there are a wide variety of applications for cell therapy, significant obstacles exist to effective cell therapy. For example, in the case of cardiovascular cell therapy, it has been estimated that only a very small percentage (i.e., from 1% to 10%) of transplanted cells survive within myocardial tissue, with most cells being lost very early after delivery. Several causative factors appear to be involved in this low cell survivability, including physical strain during injection, inflammation, apoptosis, ischemia, and lack of cell retention. Investigation has also revealed that cell settling is a significant problem, and it has been demonstrated that fibroblasts and myoblasts become significantly stratified in vials and syringes in under 30 minutes. The rapid settling of cells can create a number of problems, such as a decreased and unpredictable number of cells being delivered via techniques such as catheter delivery.

In order to provide a sufficient mass of cells for effective cell therapy, a sufficient number of cells should be delivered to the target tissue, a significant portion of the cells should remain viable, and the cells should be encouraged to remain in the target tissue. As cell settling and delivery stress both have adverse effects on providing a sufficient mass of cells, a method for delivering cells that avoids cell settling and delivery stress is needed.

### SUMMARY OF THE INVENTION

The invention provides compositions and methods for the delivery of cells to a tissue site in a subject. The composition includes a polymer that results in shear thinning properties for the composition, which can reduce cell settling and facilitate the delivery of cells. Cell settling can be reduced by using a high viscosity gel, but this generally makes cell delivery more difficult, requiring the application of higher pressure to deliver cells. This principle is illustrated by FIG. 1, which shows the increased force necessary to deliver cells through a 1 meter long, 1 millimeter (mm) internal diameter catheter at a rate of 10 milliliters (ml)/minute as the viscosity of the composition is increased. However, note that particular shear thinning character may vary considerably from that shown in FIG. 1 when different conditions are used. The problem of how to reconcile the need for viscosity at rest with the need for safe delivery of cells is overcome by the present invention. Specifically, the present invention provides a shear thinning polymer solution that has a significant viscosity when at rest, but lower viscosity when shear force is applied.

The methods and compositions of the invention can provide one or more of the following advantages. For example, the invention can reduce cell settling during delivery of cells to a tissue site. The invention can also be used to prevent cell settling during storage prior to delivery. The invention can also provide a relatively uniform distribution of cells within a particular volume, or over a period of delivery time. The invention can also promote cell viability by reducing cell stress during storage, delivery, and within tissue, and by providing a biocompatible environment. The invention can also provide for higher retention of cells in a target tissue site due to the significant viscosity of the suspension under normal in vivo conditions. The invention can also reduce and preferably eliminate the need to calibrate the delivery composition based on the nature of the cells being delivered and/or the need to include mixing devices that resuspend cells as part of the delivery system. The invention can also expand the choice of suitable catheters for delivery due to the shear thinning nature of the polymeric suspension, and/or allow the delivery of cells at reasonable pressures and/or flow rates.

Thus, in one aspect, the present invention provides a cell delivery composition that includes a biocompatible carrier liquid, a biocompatible shear thinning polymer at a concentration from greater than or equal to the shear thinning polymer's overlap concentration in the biocompatible carrier liquid up to 10 percent by weight (wt-%) concentration of the shear thinning polymer in the biocompatible carrier liquid, and a plurality of cells. In one aspect, the shear thinning polymer has a molecular weight of 1,000,000 grams per mole (g/mol) or more and is present at a concentration of 2 wt-% or less in the biocompatible carrier liquid. Weight percent of compositions including the polymer in the biocompatible carrier liquid are calculated herein by comparing the weight of the polymer to the weight of the biocompatible carrier liquid plus the polymer.

A shear thinning polymer provides a composition that exhibits shear thinning properties when placed in the carrier liquid at an appropriate concentration. Various polymers are suitable for use in cell delivery compositions of the present invention. For instance, the shear thinning polymer may be a poly(alkylene oxide) polymer. In a further embodiment, the poly(alkylene oxide) polymer is selected from the group consisting of poly(ethylene oxide), poly(propylene oxide), and poly(ethylene-co-propylene oxide) copolymers, or in a further aspect, the shear thinning polymer may specifically be poly(ethylene oxide). When poly(ethylene oxide) (PEO) is used, the poly(ethylene oxide) may be present in one embodiment at a concentration of 0.1 wt-% to 2.0 wt-% in the biocompatible carrier liquid. In a further embodiment, PEO with a molecular weight of 1,000,000 g/mol or more may be used, while an additional embodiment uses PEO at a molecular weight of 8,000,000 g/mol or more.

In one aspect, the cells provided by the invention may be selected from the group consisting of islet cells, stem cells, hepatocytes, chondrocytes, osteoblasts, neuronal cells, glial cells, smooth muscle cells, endothelial cells, nucleus pulposus cells, epithelial cells, myoblasts, myocytes, macrophages, purkinje cells, erythrocytes, platelets, fibroblasts, and combinations thereof. In a further aspect, cells suitable for the regeneration of cardiac tissue are provided. In an additional aspect, imaging or tracking agents, or polypeptides, may also be included in the composition. If a polypeptide is included, the polypeptide may be a buffering protein or a growth factor. In a further embodiment, the polypeptide may be selected from the group consisting of PDGF, VEGF, FGF, EGF, IGF, TGF-beta, MGF, cytokines, prostaglandins, collagens, elastin, fibronectin, laminin, tenascin, entactin, fibrinogen, fibrin, heparin, heparin sulfate, dermatan sulfate, keratin sulfate, and chondroitin sulfate.

Aspects of the composition used in the method may provide particular characteristics. For instance, in one aspect, the cells have a settling rate of 1 millimeter per hour or less in the cell delivery composition when it is not subjected to shear stress. In an additional aspect, the cell delivery composition exhibits a one order magnitude decrease in viscosity when the shear rate is increased from 1 s⁻¹ to the shear rate typical for cell delivery, e.g. 1000 s⁻¹. Aspects of the method may also encourage retention of cells at the tissue site to which they are delivered. For instance, the cells may be retained at the tissue site for at least an hour. In further embodiments, the cells are be retained at the tissue for at least 24 hours, or more preferably, the cells are retained at the tissue site for at least 48 hours.

In another aspect, the invention provides a method of delivering cells to a subject that includes providing a cell delivery composition and delivering the cell delivery composition to a tissue site in the subject. The cell delivery composition includes a biocompatible carrier liquid, a biocompatible shear thinning polymer at a concentration from greater than or equal to the shear thinning polymer's overlap concentration in the biocompatible carrier liquid up to 10 wt-% of shear thinning polymer in the biocompatible carrier liquid, and a plurality of cells. In a further aspect, the shear thinning polymer used in the method has a molecular weight of 1,000,000 g/mol or more and is present at a concentration of 2 wt-% or less in the biocompatible carrier liquid.

Again, a variety of polymers are suitable for use in the method. In one aspect, the shear thinning polymer is a poly(alkylene oxide) polymer. In a further aspect, the poly(alkylene oxide) polymer is selected from the group consisting of poly(ethylene oxide), poly(propylene oxide), and poly(ethylene-co-propylene oxide) copolymers. In an additional aspect, the shear thinning polymer is a poly(ethylene oxide). When the shear thinning polymer is PEO, in further aspects it may be present at a concentration of 0.1 wt-% to 2.0 wt-% in the biocompatible carrier liquid. In further aspects, PEO with a molecular weight of 1,000,000 g/mol or more may be used, or more preferably a molecular weight of 8,000,000 g/mol or more.

In an additional aspect of the method of delivering cells, the cells are selected from the group consisting of islet cells, stem cells, hepatocytes, chondrocytes, osteoblasts, neuronal cells, glial cells, smooth muscle cells, endothelial cells, nucleus pulposus cells, epithelial cells, myoblasts, myocytes, macrophages, purkinje cells, erythrocytes, platelets, fibroblasts, and combinations thereof. In a further aspect, the cell concentrations may be from 1×10⁶ cells per milliliter to 1×10⁹ cells per milliliter of the cell delivery composition.

Aspects of the method of delivering cells may utilize compositions that provide particular characteristics. For instance, in one aspect, the cells have a settling rate of 1 millimeter per hour or less in the cell delivery composition when it is not subjected to shear stress. In a further aspect, the cell delivery composition exhibits a one order magnitude decrease in viscosity when the shear rate is increased from 1 s⁻¹ to 1000 s⁻¹. In an additional aspect, the method of delivering the cells further includes delivering the cell delivery composition through a catheter.

Methods of delivering cells to a subject include delivering the cell delivery composition to particular tissue sites. For instance, the tissue site may include epithelial, connective, skeletal, muscular, glandular, or nervous tissue. A preferred tissue site is cardiac tissue. In an additional aspect of the method, the subject may be a mammal, and in a further aspect the mammal may be a human. In a preferred aspect, 70% or more of the cells remain viable after delivery to a tissue site. In a further aspect, the cells are delivered to the tissue site at a constant rate.

Another aspect of the invention includes a method of cardiovascular regeneration that includes providing a cell delivery composition that includes a biocompatible carrier liquid, a poly(ethylene oxide) polymer with a molecular weight of 1,000,000 g/mol or more at a concentration of 0.1 wt-% to 2.0 wt-% in the biocompatible carrier liquid, and a plurality of mammalian cells suitable for cardiovascular application, and delivering the cell delivery composition including the mammalian cells at a constant rate to a cardiac tissue site in a mammal, wherein 70% or more of the mammalian cells remain viable after delivery to the cardiac tissue site.

The terms “comprises” and variations thereof do not have a limiting meaning where these terms appear in the description and claims.

The term “alkyl,” as used herein, refers to a saturated hydrocarbon group typically although not necessarily containing 1 to 30 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to 12 carbon atoms. The term “lower alkyl” intends an alkyl group of 1 to 6 carbon atoms, preferably 1 to 4 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl groups, respectively.

Unless otherwise specified, “alkylene” is the divalent form of the “alkyl” group defined above. The term “alkylenyl” may be used when “alkylene” is substituted. For example, an arylalkylenyl group comprises an alkylene moiety to which an aryl group is attached. Accordingly, the term “alkylene oxide,” as used herein, refers to a divalent form of an alkyl group including an oxygen substituted for a hydrogen atom. For example, an alkylene oxide in which the alkyl group is an ethyl group is ethylene oxide.

Unless otherwise specified, “a,” “an,” “the,” and “at least one” are used interchangeably and mean one or more than one. Thus, for example, a composition that comprises “a” type of cell can be interpreted to mean that the composition includes “one or more” types of cells. Similarly, a composition comprising “a” polymer can be interpreted to mean that the composition includes “one or more” polymers.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “room temperature” refers to a temperature of 20° C. to 25° C. or 22° C. to 25° C.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a graph showing the reciprocal relationship of cell settling and delivery force in a non-shear thinning composition of varying viscosity.

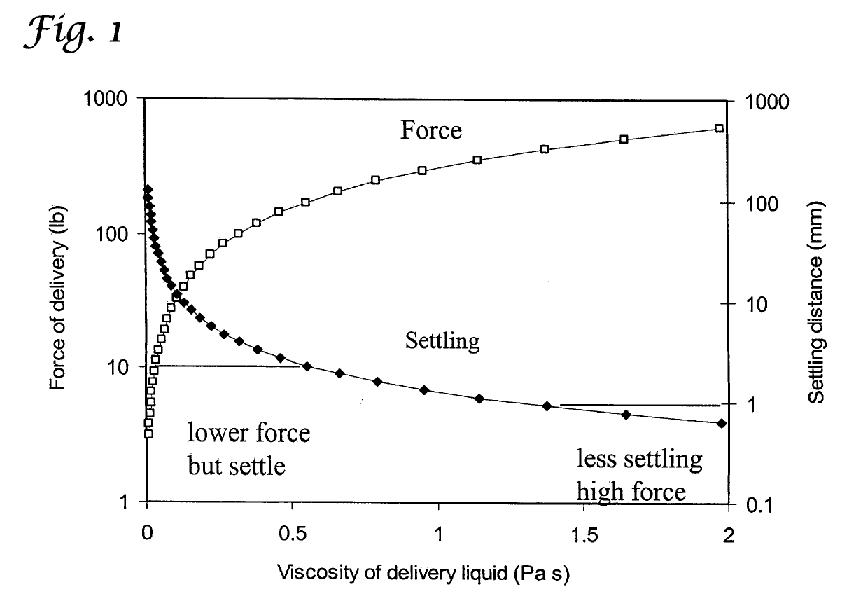

FIG. 2 is a graph showing the changes of UV absorption at 300 nm as a function of time for different polymeric cell carriers.

FIG. 3 is a bar graph showing the cell concentrations at 0 minutes and 60 minutes after automated delivery through a catheter in polymer solutions (PEO and PVP) and plain buffer solution (HBSS).

FIG. 4 is a graph showing the cell settling in PEO solutions of various molecular weights over time.

FIG. 5 is a graph showing the viscosity (in Pa s) of PEO/buffer solution versus PEO concentration.

FIG. 6 is a graph showing the viscosity of (in Pa s) of five PEO/buffer solutions with various molecular weights versus the Wt % of the PEO in buffer solution.

FIG. 7 is a graph of the steady shear viscosity (Pa s) versus shear rate (s⁻¹) of a PEO/buffer solution.

### DETAILED DESCRIPTION OF ILLUSTRATIVE EMBODIMENTS OF THE INVENTION

The invention provides compositions and methods for the delivery of viable cells to a tissue site in a subject. In one aspect, the invention provides a cell delivery composition that includes a biocompatible shear thinning polymer in a biocompatible carrier liquid. A shear thinning polymer solution has a higher viscosity when at rest or when subject to slower shearing, but a lower viscosity when subjected to a higher shear rate. A shear thinning material will also generally return to a viscosity at or near its previous resting state viscosity upon removal of the shear stress. Generally, the viscosity of a shear thinning polymer solution varies inversely, in a non-linear fashion, with the level of force applied.

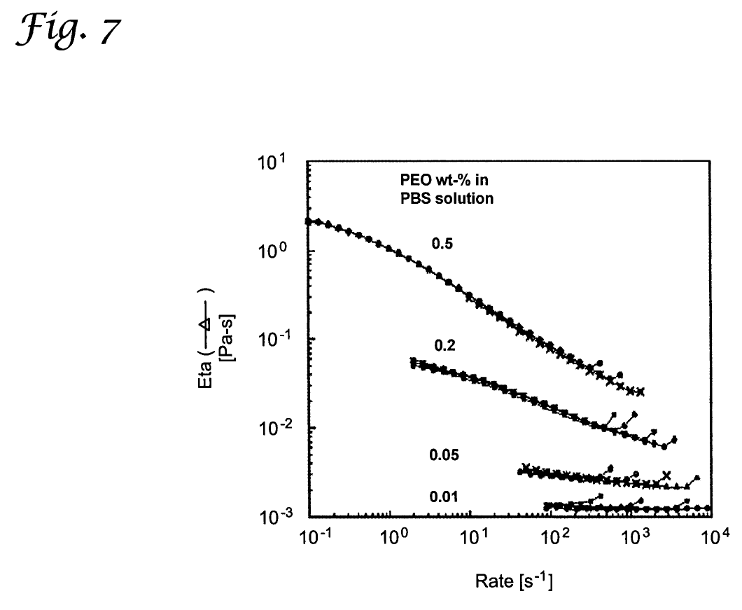

The inverse relationship between the shear rate (\dot{γ}) and viscosity (η) is typically nonlinear and can be described with the following equation,
η=η_{S}·\dot{γ}ⁿ⁻¹
where η_{S} is the viscosity coefficient and n is the so called non-Newtonian index that measures how far the flow behavior of a material deviates from that of a Newtonian fluid whose viscosity is independent of shear rate, e.g. water. Newtonian fluids have a value of n=1, while shear thinning materials have a value of n<1. For polymer materials and their solutions, n is a number that ranges from 0.1 to 1. For a shear thinning material, n can decrease with increasing shear rate. For example, for a solution of PEO in water, n is about 0.4 to 0.7 when PEO concentration is from 0.2 wt-% to 0.5 wt-% and the shear rate is from 10 s⁻¹ to 1000 s⁻¹. See FIG. 7 for examples of the effect of shear rate on viscosity at various concentrations.

While not intending to be bound by theory, the viscosity of solutions with polymers that confer shear thinning properties (i.e., shear thinning polymers) arises from the re-orientation, alignment, and/or disentanglement (i.e., release from hindered states) of polymer chains upon application of shear stress (e.g., mechanical force). The greater the length of the chains, the more movement is hindered and the higher the viscosity of a solution containing the shear thinning polymer. Normally, polymer chains exist as random coils. However, when subject to high shear stress, the chains align themselves in a more parallel fashion, resulting in a decrease in viscosity. When polymer chains are large, they tend to entangle with each other to a greater extent. As long as entanglement occurs, the viscosity of polymers increases dramatically with increasing molecular weight. However, when shear stress is applied to the entangled polymer, the polymer chains can become partially or fully disentangled, resulting in a decrease in viscosity. The shear thinning phenomena is explained by these two reasons; however, it may be linked to other mechanisms as well.

Shear thinning occurs not only in bulk polymers but also in polymer solutions. When polymers are dissolved or suspended in solvent, the individual chains typically form swollen coils. The long polymer chains of shear thinning polymers form loosely packed coils in which the radius of the coils is proportional to the number of monomers per chain. This is more precisely expressed as the radius being proportional to N^{ν}, where N is the number of monomers per chain, and the exponent ν expresses an interaction between the polymer and the solvent that effectively increases the volume occupied by the packed coil. This second, “virtual” volume, representing the polymer in addition to a region of solvent interacting with the polymer, is referred to as the coil volume. Shear thinning polymers of the present invention are preferably water-soluble at the temperature that they are going to be used, which is typically room or body temperature. Preferably, ν>0.5, more preferably, ν=0.6.

At low concentration, the coils are isolated from each other, but they start to contact each other when the concentration of polymer reaches a value called the contact or overlap concentration. Before the overlap concentration is reached, the viscosity of the solution can be described with the Einstein Equation:
η=η₀·(1+2.5φ_{c})
where η₀ is the viscosity of solvent and φ_{c} is the volume concentration of the chain coils. Generally, no shear thinning is observed at low concentrations in solution. After the overlap concentration is reached, the Theological behavior of solution changes; the viscosity-concentration relationship is no longer linear and shear thinning begins to occur. At the overlap concentration, the solution reaches a state where φ_{c} appears to be 100%, and the solution begins to exhibit shear thinning viscosity due to coil entanglement. Polymers that occupy a significantly greater coil volume are more likely to contact and overlap one another. A preferred shear thinning polymer is thus a polymer that has a sufficient number of monomeric units, and a sufficient level of interaction with the solvent, to result in shear thinning viscosity in solution at relatively low concentrations.

As the concentration of polymer coils in a solution increases, a point is reached where, on average, they just begin to overlap. This concentration is known as the “overlap concentration.” The overlap concentration c*, can be calculated from:
c*=N/R_{g}³
where N is the degree of polymerization and R_{g} is the coil size. Further information on the calculation of overlap concentrations for polymers is provided, for example, by Gennes (Gennes, P., Scaling concepts in polymer physics, Ch. 2, Cornell University Press (1979), Doi (Doi, M., Introduction to Polymer Physics, Ch. 2, Clarendon Press (1996)), and Strobl (Strobl, G., The Physics of Polymers, 2^{nd} ed., Ch. 2, Springer Press (1997)).

The volume concentration of chain coils is different from the polymer concentration in solution. Polymer chains interact with themselves and solvent molecules. Also, maximization of entropy drives polymer chains to take a random walk conformation (Gaussian distribution) in space. As a result, polymer chains expand and occupy a much larger space than their own volume. For example, for a polymer chain with N monomers of a size a, the polymer chain's own volume is Na³, but its coil volume is proportional to N^{3ν}ₐ³, where ν=0.6 for polymers in “good solvents” and 0.5 in “θ solvents” (where the polymer chain takes the conformation of an ideal chain). Note that the terms “good solvent” and “θ solvent” are used herein according to their definition in the polymer arts (See Pierre-Gilles de Gennes, Scaling concepts in polymer physics, Cornell University Press, Ithaca N.Y., 1979). Thus, the coil volume may be N^{0.5} to N^{0.8} times greater than its own volume. The volume fraction of the chain coil is thus much greater than the polymer volume concentration. For example, for a solution of PEO in water, if ν=0.5 and molecular weight=8M g/mol (N=182 K), then the coil volume is 426 times larger than polymer volume. If ν=0.6, the coil is 16133 times larger. Typically, v is between 0.5 and 0.6, and the chains reach the contact or overlap concentration at a concentration from 0.01 wt-% to 0.3 wt-%. Therefore, a solution of PEO in water can exhibit high viscosity and shear thinning behaviour at very low concentrations.

For the present application, preferably, a shear thinning polymer solution exhibits a one order magnitude decrease in viscosity when the shear rate is increased from 1 inverse second to 1000 inverse seconds (s⁻¹). More preferably, the polymer solution exhibits a two order magnitude decrease in viscosity when the shear rate is increased from 1 s⁻¹ to 1000 s⁻¹.

Rate dependent viscosity may also be observed in “thixotropic” materials. A thixotropic material is typically a particulate suspension in solvent, often one that forms a colloid. For example, whipped cream is a thixotropic material. Particles of thixotropic material can aggregate into large structures, leading the suspension to have a high viscosity, and may even provide some structural strength. When subject to mechanical force, the aggregates generally break down and the viscosity of the suspension decreases dramatically. The broken structures can re-aggregate, resulting in recovery of high viscosity, but the process is time dependent, which is one difference from what occurs with a shear thinning polymer solution. Thixotropic fluids generally become less viscous as a function of time, again in contrast with the shear thinning compositions of the present invention.

As shear thinning behavior generally appears after the overlap concentration has been reached or exceeded, the shear thinning polymer of the invention is preferably present in the cell delivery composition at a concentration greater than or equal to its overlap concentration. It is also preferred that the shear thinning polymer of the invention not exhibit cross-linking, as this will diminish the mobility of the polymer particles that is needed for shear thinning behavior.

Shear thinning polymers used in the cell delivery composition of the present invention should be biocompatible. A biocompatible material, as used herein, refers to a material that produces little if any adverse biological response when used in vivo. Biocompatibility is achieved as a result of the nature of the polymer itself, or through the ability of the polymer to be effective at sufficiently low concentrations to reduce an adverse biological response, or through a combination of the two. Preferably, degradation products of the shear thinning polymer are biocompatible as well.

It is also preferable for the shear thinning polymers to have high molecular weight. As described above, high molecular weight is one factor causing chain coils to overlap and entangle with each other, resulting in shear thinning behavior. High molecular weight polymers provide at least two advantages in terms of biocompatibility. First, high molecular weight polymers more readily exhibit shear thinning viscosity due to coil entanglement, and hence can be used at a lower concentration. Furthermore, high molecular weight polymers generally exhibit a lower osmotic effect on cells, as cells are better able to exclude material with a high molecular weight. As the osmotic effect can lead to swelling of the cell and other toxic effects due to polymer uptake, it is preferable for shear thinning polymers of the invention to minimize osmotic effects. Preferably the shear thinning polymers also have a high affinity for their solvent (e.g., water) and are relatively linear. A combination of these attributes is preferred, and preferably provides a shear thinning polymer that is effective at a very low concentration.

Shear thinning polymers of the invention exhibit shear thinning behavior when dissolved in a carrier liquid at a concentration greater than or equal to the polymer's overlap concentration. Shear thinning polymers of the invention should therefore be soluble in the carrier liquid. Solubility, as defined herein, is used in the broader sense of solubility, and refers to the ability of the shear thinning polymers to blend uniformly with the carrier liquid, and includes material that is uniformly suspended. True solubility, in which the shear thinning polymer forms a uniformly dispersed mixture at the molecular or ionic level in the carrier liquid, is not required for the invention. A polymer solution is a carrier liquid including a polymer that is soluble in the carrier liquid.

Shear thinning polymers of the present invention are preferably used at concentrations low enough to provide biocompatibility and to avoid formation of a thick gel. Thus, the shear thinning polymers of the present invention are preferably used at a concentration of 10 wt-% or less in the biocompatible carrier liquid.

As increased cell survival is an important aspect of the invention, the carrier liquid used in the composition will typically be an aqueous solution. Mixing of the shear thinning polymer with the carrier liquid can be achieved, for example, with conventional low shear methods. Shear thinning behavior is exhibited by the shear thinning polymers at relatively low concentrations in solution. Preferably, the shear thinning polymer has a concentration of 2.0 wt-% or less in solution, in relation to the carrier liquid and the polymer. More preferably, the shear thinning polymer has a concentration of 1.0 wt-% or less. In further aspects of the invention, the shear thinning polymer has a concentration of 0.5 wt-% or less, or 0.2 wt-% or less. Viscosity of the shear thinning polymer solution when subject to shear during deliver of cells is preferably lower than 0.05 Pa s, and more preferably lower than 0.01 Pa s, and even more preferably lower than 0.005 Pa s.

In one aspect of the invention, the shear thinning polymer is poly(ethylene oxide. See, for instance, Examples 3-5, herein. When using PEO, it is preferable to use a polymer that has a molecular weight of 1,000,000 grams/mole (g/mol) or more, preferably, 4,000,000 g/mol or more, and even more preferably, 8,000,000 g/mol or more. High molecular weight PEO has a virtual volume that is many times that of its actual volume, greatly increasing its ability to reach overlap concentration at relatively low polymer concentrations in solution. Preferably, the PEO is a linear molecule; however, PEO with significant branching and side chains may also be used.

In a further aspect of the invention, the shear thinning polymer may be a poly(propylene oxide) (PPO). Random and block copolymers of PEO and PPO (PEO-PPO) may be used to form poly(ethylene-co-propylene oxide) with various ratios of ethylene to propylene. For instance, the amount of ethylene may range from 5% to 95%, with the remainder consisting of propylene. PPO and PEO-PPO copolymers are also preferably linear, but may also be used when they include significant branching and side chains.

A variety of polymers are suitable for use in the present invention. For example, shear thinning polymers may include poly(alkylene oxide) polymers, or more preferably poly(alkylene oxide) polymers wherein the alkyl group is a lower alkyl group. In another aspect, the shear thinning polymer can be polyacrylamides or ionized polymers (e.g. sulfonated polystyrene). The shear thinning polymer solution may also include a combination of more than one polymer. Shear thinning polymers can also be those natural polymers such as polysaccharides and their derivatives, DNA, proteins, and combinations thereof, so long as the molecules of the shear thinning polymer exhibit the shear thinning characteristics described herein. A shear thinning polymer preferably has a high molecular weight (e.g., a MW of 1,000,000 g/mol or more). Shear thinning polymers of the invention are preferably linear molecules, or molecules with a limited amount of branching and/or sidechains, and are not crosslinked. Shear thinning polymers are also preferably shear thinning at concentrations of 2.0 wt-% or less, and are soluble in aqueous solutions.

The cell delivery composition of the invention includes a shear thinning polymer in a biocompatible carrier liquid. The carrier liquid should be biocompatible to reduce undesirable effects on the delivered cells or the subject to which they are delivered. Biocompatibility of the carrier liquid is defined in the same fashion as it is for polymer, herein. The carrier liquid may be an aqueous buffer or a tissue culture media, or a combination of the two. An aqueous buffer solution is a buffer solution based on water. A wide variety of biocompatible aqueous buffer solutions are available and known to those skilled in the art. The choice of aqueous buffer used will vary depending on the needs of the mammalian cells being delivered. For a variety of buffers and tissue culture media, see the 2005 Sigma Biochemicals and Reagents catalog (SIGMA-ALDRICH Company). Typically, a buffer includes one or more salts, dissolved in a sterile water solution, that are chosen to maintain the pH of the solution within a particular range. The biocompatible carrier liquid may also include growth-related substances such as preservatives, nutrients, antibiotics, or other compounds useful to sustain viable cells. Should sufficient quantities of these growth-related substances be present, the liquid will generally be categorized as a tissue culture media, rather than an aqueous buffer. Preferred carrier liquids for use with fibroblasts and/or myoblasts, delivered in embodiments of the invention further described herein, include Phosphate Buffered Saline (PBS), a well-known buffer made up from KH₂PO₄, K₂HPO₄, and NaCl dissolved in aqueous solution, and Hanks Balanced Salts Solution (HBSS), a more complex mixture of predominantly NaCl, Glucose, KCl, and NaHCO₃ in aqueous solution, that is generally categorized as a tissue culture media.

The cell delivery composition may also include polypeptides. As used herein, the term “polypeptide” refers broadly to a polymer of two or more amino acids joined together by peptide bonds. The term “polypeptide” also includes molecules that contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring. Peptides may be included to provide a buffering capability or to promote cell survival and growth in other ways. For example, albumin may be included in the cell delivery composition to serve as a buffer, whereas various growth factors may be included to promote angiogenesis, cell growth, or retention in the tissue site.

Examples of polypeptides that may be included in the cell delivery composition include growth factors involved in cell proliferation, migration, differentiation, cell signaling such as PDGF (platelet derived growth factor), VEGF (vascular endothelial growth factor) and its family of proteins, FGF (fibroblast growth factor), EGF (epidermal growth factor), IGF (insulin like growth factor), TGF-beta (transforming growth factor), and NGF (neurotropic growth factor), etc.). Other polypeptides include cytokines, prostaglandins and extracellular matrix (ECM) proteins (including structural ECMs such as collagens I, II, III, IV and elastin; adhesion ECMs such s fibronectin, laminin, tenascin, entactin, fibrinogen, and fibrin; and proteoglycans such as heparin and heparan sulfate, dermatan sulfate, keratan sulfate, and chondroitin sulfate). Further polypeptides include enzymes, enzyme inhibitors such as TIMPS (tissue inhibitors of matrix metalloproteinases), antibodies, and protein derivatives such as gelatin. Polypeptide mixtures and/or combinations either involving a few selected proteins or a combination of many factors such as serum-derived proteins or serum itself may also be provided.

The cell delivery composition also includes a plurality of cells, preferably mammalian cells. The cell delivery composition may include a single type of cell, or it may include various different types of cells. Preferably, the cells are suspended in a dispersed fashion within the shear thinning material, which is a shear thinning polymer dissolved in a biocompatible carrier liquid. Cells can be obtained directly from a donor, or from established cell lines. Examples of such cells include mature myogenic cells (e.g., skeletal myocytes, cardiomyocytes, purkinje cells, and fibroblasts), progenitor myogenic cells (e.g., myoblasts), mature non-myogenic cells (e.g., endothelial and epithelial cells), hematopoietic cells (e.g., monocytes, macrophages, fibroblasts, (x-islet cells, β-islet cells, cord blood cells, erythrocytes, and platelets) and stem cells (e.g., pluripotent stem cells, mesenchymal stem cells, endothermal stem cells, ectodermal stem cells). More particularly, cells that may be included in the cell delivery composition of the invention include islet cells, hepatocytes, chondrocytes, osteoblasts, neuronal cells, glial cells, smooth muscle cells, endothelial cells, skeletal myoblasts, nucleus pulposus cells, and epithelial cells.

Preferred cells include cells that are suitable for cardiovascular applications. Particularly preferred are those cell subtypes with potential regenerative capacity. Sources and types of cells suitable for cardiovascular application include bone marrow, which can provide mononuclear cells, stromal cells, CD34⁺ cells, CD133⁺ cells, and endothelial cells; peripheral blood, which can supply endothelial progenitor cells, umbilical cord blood, which can provide CD34⁺ cells, CD133⁺ cells, multipotent adult progenitor cells, and somatic stem cells; adipose tissue, which can provide stromal cells and CD34⁺ cells; skeletal muscle, which can provide skeletal myoblasts and skeletal muscle stem cells; and cardiac muscle, which can provide cardiac stem cells. Embryonic stem cells may also be considered a source of cells useful for cardiovascular applications.

The cell types may be autologous, allogenic, or xenogenic. Preferably, the cells used are from the same species, and have a compatible immunological profile, evaluated by analysis of cells obtained by biopsy, either from the subject or a close relative. Autologous cells are preferred, as they do not provoke an immune reaction, they provide a minimal risk of anaphylaxis, transfusion reactions, and alloimmunization, they provide a reduced risk from transmissible infectious agents, and they provide rapid access to large numbers of cells (e.g., post-mobilization leukapheresis product). However, allogeneic cells are not without advantages, as they are often immediately available “off the shelf” in large numbers, they provide greater access to genetically modified cells, they allow various supplemental steps such as bone marrow aspirate, cytokine mobilization, and skeletal muscle biopsy may be avoided, and cells from young donors may overcome issues of age-related decline of regenerative capacity. If cells are used that may elicit an immune reaction, such as cells from an immunologically distinct donor, then the recipient of the cells can be immunosuppressed as needed, for example using a schedule of immunosuppressant drugs such as cyclosporine.

Cells used in the invention may also be genetically engineered by viral or non-viral means, using methods that are readily known by those skilled in the art. For example, cells may be genetically engineered to secrete survival or growth factors. Also, cells of different types may be included in a single composition. For example, a single composition could contain both fibroblasts and myoblasts. As the cells function in the invention primarily as an item being delivered by the cell delivery composition including the shear thinning polymer, the particular species of cells being delivered may vary considerably. The nature of the cells being delivered is primarily of importance only with regard to nature of the tissue site to which they are being delivered, and the type of therapy that the cells are intended to facilitate.

The number of cells contained within the cell delivery composition may vary considerably. For instance, preferred cell concentrations may be as high as 1×10⁹ cells per milliliter (ml) of the cell delivery composition. Alternately, preferred cell concentrations may be as low as 1×10⁶ cells per ml of the cell delivery composition. While the concentrations listed are preferred, as it is generally desirable to provide a substantial number of cells to a particular tissue site, the invention also includes the cell delivery compositions containing lower numbers of cells. The concentration of cells within a volume of the cell delivery composition may depend to some extent on the size of the cells being delivered.

In order to track the delivery of a cell delivery composition and the cells it contains to a tissue site, as well as what happens to the composition after delivery, it may be preferable to include imaging and/or tracking agents within the cell delivery composition. These imaging and/or tracking agents may be included in the liquid (e.g., aqueous) portion of the composition, the shear thinning polymer, or the actual cells themselves. Imaging and/or tracking reagents include iodine-based solutions such as iopamidol, as well as other agents such as gadodiamide and iron dextran. Cells or reagents may also be fluorescently labeled or genetically marked with green fluorescent protein or LacZ for beta-galactosidase detection, or labeled with radioactive elements (e.g., C¹⁴, H³, 111In-oxine, or I¹²⁵) to facilitate their tracking through the radioactive tag, using methods known to those skilled in the art. Additionally, stable isotopes such as nano-size Europium particles can be used that become radioactive following neutron activation. Additionally, cells can carry nano-sized paramagnetic iron oxide particles for MRI detection.

The invention also provides methods for using a cell delivery composition to deliver cells to a tissue site in a subject. The methods include providing a cell delivery composition, as described herein, that includes a biocompatible shear thinning polymer, a biocompatible carrier liquid, and a plurality of cells. The method further includes delivering these cells to a tissue site in a subject. Preferably, the cells being delivered are mammalian cells.

Preferably, the cells are reliably delivered to the tissue site by the cell delivery composition. Reliable delivery of cells to a tissue site in a subject includes delivery in which a reasonably predictable number of cells are delivered to a particular site within an organism. More preferably, reliable delivery of cells to a tissue site in a subject includes delivery in which not only a predictable number of cells are delivered, but the cells are further delivered at a predictable rate. Reliable delivery of cells is facilitated by the cell delivery composition of the invention due, in part, to the ability of the cell delivery composition to retain cells in suspension, in a relatively even dispersion, over a significant period of time. By resisting motion of the cells, the shear thinning polymer prevents settling of the cells, generally due to the force of gravity. Preferably, cells in a cell delivery composition of the invention settle at a rate of 1 mm per hour or less. By retaining an even dispersion of cells within the cell delivery device (e.g., a syringe), expulsion of portions of the cell delivery composition to the tissue site results in a constant number of cells being delivered by a given portion, as all portions within the cell delivery device will contain an essentially equivalent number of cells. So long as the rate and volume of the portions being delivered are kept relatively constant, this will also result in a constant rate of delivery of a constant number of cells, making delivery of cells more predictable.

In a further aspect, reliable delivery of the cells includes the delivery of cells that remain viable. Cells remain viable, as defined herein, by retaining the capacity to perform one or more of the following functions, such as metabolism, growth, reproduction, or some form of responsiveness. The extent and character of these signs of viability will vary from one cell type to another, as known by those skilled in the art. Cell viability may be readily evaluated using techniques known to those skilled in the art. For example, cell viability may be evaluated by visual observation with a light or scanning electron microscope, histology (e.g., trypan blue staining) or quantitative assessment with radioisotopes. Cell viability is provided by the cell delivery compositions of the invention by reducing stress (e.g, mechanical stress) on the cells before, after, and during delivery, and by providing a biocompatible environment that reduces hazards to the cell such as osmotic pressure. Preferably, at least 70% of the cells suspended in a cell delivery composition of the invention remain viable within an hour after delivery. More preferably, at least 90% of the cells suspended in a cell delivery composition of the invention remain viable within an hour after delivery.

The invention provides a method of delivering cells to a subject. As used herein, a “subject” is an organism, including, for example, an animal. This includes, for example, humans, nonhuman primates, sheep, horses, cattle, pigs, dogs, cats, rats, mice, birds, reptiles, fish, insects, arachnids, protists (e.g., protozoa), and prokaryotic bacteria. Preferably, the subject is a mammal. Mammals are a vertebrate class of animals that includes the subclasses of marsupials, monotremes, and placental mammals, with the majority of mammals being placental mammals. The subclass of placental mammals in divided into various orders. Within these orders are included various domesticated animals such as cats, dogs, cows, sheep, goats, pigs, and horses. Also included are various mammals commonly used as laboratory animals, such as rodents and primates. A preferred class of mammals for use in the method are humans.

The invention also provides a method of delivering cells to a tissue site in a subject. A tissue site is a particular location within the body of the subject where cells are delivered and preferably retained. Tissue, as defined herein, is a part of an organism made up of an aggregate of cells having a similar structure and function, or functions that that can work together. Tissue is generally divided into parenchyma, which is the tissue that forms an organ, and the stroma, which is tissue that supports the organs. Tissues include epithelial, connective, skeletal, muscular, glandular, and nervous tissues. Tissue sites may also be defined by their function; for example, blood vessel, cardiac, lung, and brain tissue may all be tissue sites for delivery of cells by the cell delivery composition of the invention. A preferred tissue is cardiac tissue, which further includes the myocardium, papillary muscle, SA node, atrioventricular node, atrioventricular bundle, and the purkinje network tissue sites. Cells delivered to a tissue site in a subject are preferably retained at that tissue site in sufficient numbers to generate the desired result (e.g., initiation of tissue regeneration).

The cell delivery composition of the invention exhibits shear thinning viscosity, and hence will encourage delivered cells to remain within the cell delivery composition at the tissue site, as viscosity of the composition increases when the composition is not subjected to shear stress. For instance, cells may be retained at the tissue site for at least an hour after delivery. In additional embodiments of the invention, cells may be retained at the tissue site for at least 24 hours, or, more preferably, for at least 48 hours. While not intending to be bound by theory, cells related to the cells present at the tissue delivery site (e.g., myoblasts delivered to cardiac tissue) may further be retained in the tissue site through the activity of cell adhesion molecules such as selectins and integrins that mediate homophilic adhesion of cells of given or related types.

The cell delivery composition of the invention will typically be injected from a delivery device such as a hypodermic syringe, catheter, lead, or trocar, that has been pre-filled with the cell delivery composition. Injection through a delivery tube (e.g., needle or catheter) permits the precise administration of a desired amount of the cell delivery composition at the tissue site. The cell delivery composition may be delivered from the delivery device manually, or automatically using a pump or mechanized dispensing system. Optionally, the delivery device may include a metering device or additional device to assist in the precise delivery of the cell delivery composition. The tissue site may be accessed by the delivery device through surgical exposure, or through a percutaneous approach. When delivering cells percutaneously, various methods may be used to target cell administration, such as X-ray fluoroscopic guidance, real-time magnetic resonance imaging, or any other type of radiological guidance.

The delivery tube may be formed from various materials including, but not limited to, metallic materials, non-metallic materials, ceramic materials, polymeric materials, and composites thereof. Typical metallic materials include stainless steel, titanium, or nickel/titanium alloys, while typical polymeric materials include polyurethane, polyimide, polyetheretherketone, polysulfone, polyamides, polyethers, polyesters, polyvinyls, polyolefins, silicone, and copolymer blends and composites thereofor. Preferably, the delivery tube has a length of less than 8 feet; with a length of 3 to 6 feet typically being used. A variety of internal diameters (I.D.) within the delivery tube (e.g., needle or catheter) of the delivery device can be used. The internal diameter of the delivery tube affects the fluid dynamics of the delivered compositions, and thus the choice of I.D., can be significant. Generally, it is preferred that the smallest size I.D. possible be used, to reduce trauma and subject discomfort. However, the larger the I.D., the faster the cell delivery composition can be delivered. Thus, it is desirable to be able to inject the composition of the invention through a needle or catheter with a size of 16 gauge (0.047 inch I.D.) or smaller, preferably 20 gauge (0.023 inch I.D.) or smaller, more preferably 22 gauge (0.016 inch I.D.) and smaller, and even more preferably 24 gauge (0.012 inch I.D.) and smaller, and still more preferably 27 gauge (0.008 inch I.D.) and smaller.

A preferred tissue serving as a tissue site for the method of delivering cells is cardiac tissue. A number of methods are available for cell delivery to cardiac tissues. These methods include the use of percutaneous catheters, intracoronary infusion, transcoronary sinus retrograde infusion, endomyocardial needle injection, transcoronary vein intramyocardial injection, intrapericardial delivery, open chest transepicardial intramyocardial injection, lower limb delivery, intra-arterial infusion, and direct intramuscular injection. A variety of delivery methods for cardiovascular applications have been described (de Silva et al., Cytotherapy 6, 608-614 (2004)). The choice of delivery method can be made by one skilled in the art in light of the particular cell therapy being conducted. For example, catheter-based intramyocardial injection of autologous skeletal myoblasts for treatment of ischemic heart failure is discussed in the literature (Smits et al., J. Am. Coll. Cardiol., 42(12), 2063-2069 (2003)).

The cell delivery compositions include a shear thinning polymer solution that exhibits reduced viscosity when subjected to shear force. Accordingly, the cell delivery composition may be subjected to a shearing force when injected from a syringe or catheter that temporarily reduces the viscosity of the cell delivery composition during the injection process. Due to the decrease in viscosity upon application of shear stress, cells may be delivered more quickly and/or at a relatively low pressure, reducing stress upon the cells. The shear thinning properties of the cell delivery compositions of the invention thus allow for an effective and less invasive delivery via a needle or catheter to various sites on or within the body of a mammal. For example, as shown in Example 4 below, delivery of a 0.2 wt-% 8,000,000 g/mol MW PEO solution can be carried out at about 14 microliters/second at 160 pounds per square inch (psi) (1,100 kilopascals (kPa)), and about 4 microliters/second at 80 psi (550 kPa).

One skilled in the art will recognize that the survivability of cells in a delivery composition is proportional to the shear stress in the catheter and the length of time the cells experience the shear force. It is recognized that the effective time that a cell experiences shear stress in the needle or catheter may be as short as about 10 milliseconds, up to about 5 seconds. The survival rates for cells may be effectively improved using the cell delivery composition of the invention based on the delivery requirements, the shear stress, and the delivery time.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Evaluation of Cell Settling by UV-VIS Spectrophotometry

Human skeletal myoblasts were dispersed in different polymer solutions to achieve a final concentration of 10⁶ cells per ml. The different polymer solutions were 0.2 weight percent (wt-%) poly(ethylene oxide) (PEO), 0.2 wt-% alginate (Alg), 0.2 wt-% poly(4-vinyl pyrrolidone) (PVP), 0.5 wt-% PVP, and 1.0 wt-% PVP. All of the solutions were prepared in Hank's Balanced Salt Solution (HBSS) at pH 7.4. All of the polymers and buffers were obtained from SIGMA-ALDRICH (Milwaukee, Wis.). Cell suspension in each of the polymer solutions (0.8 ml) was then added to a disposable acrylic cuvette (10×4×45 mm, SARSTEDT), which was then covered with aluminum foil to prevent evaporation. The cuvettes were then each measured for UV absorption at 300 nanometers (nm). A constant position approximately ⅔rds of the way from the bottom of the liquid region within the cuvette was used to obtain absorbance measurements for each cuvette. The absorbance was then measured again at 45 minutes and 105 minutes after initial placement of the cells in the polymer solutions.

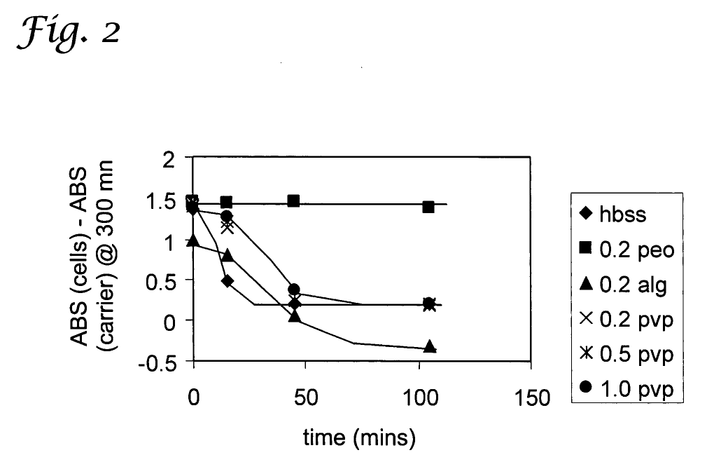

The only solution that prevented significant cell settling was the 0.2 wt-% PEO solution. The PVP solutions helped to slow down cell sedimentation, but had a much weaker effect than that of PEO, and varying the concentration of PVP present appeared to have little effect. Alginate appears to have had an effect similar to that of PVP with respect to cell settling, but this result is obscured to some extent by the sedimentation of alginate itself over the measurement period, resulting in negative absorbance readings. None of the signals other than alginate dropped to 0.0 Absorbance Units (AU); instead they stabilized at 0.2 AU, even at 105 minutes, indicating either the presence of non-settled cells or their components, or cell adhesion to cuvette walls. These results are shown in FIG. 2, which shows the absorbance of PEO, ALG, and PVP over time.

### Example 2

### Effect of Different Polymer Solutions on Cell Suspension and Viability

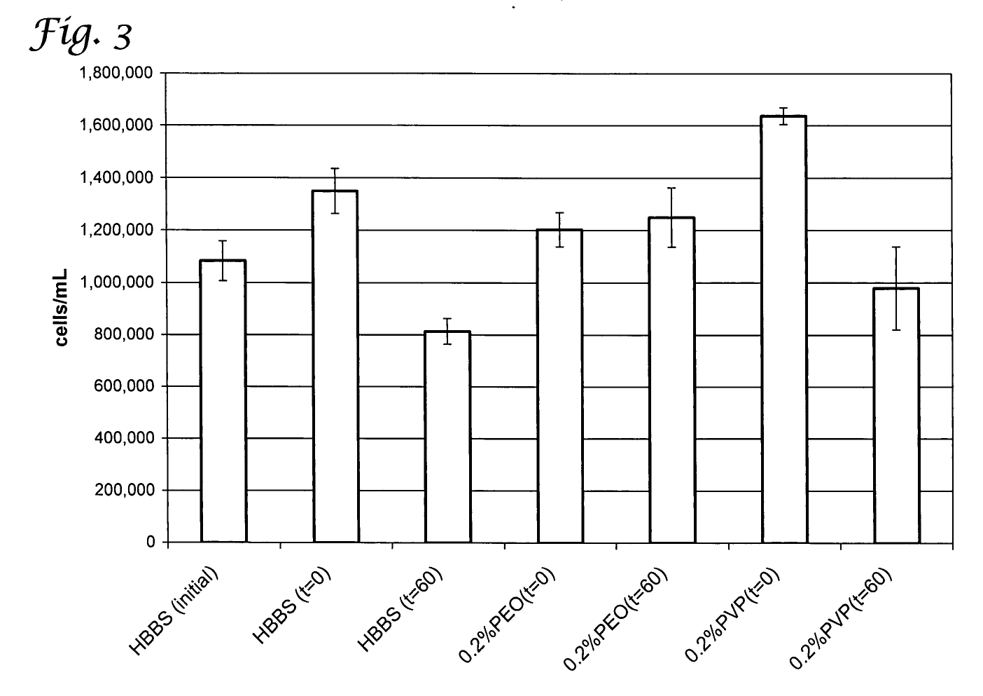

A set of experiments were conducted to evaluate the effect of polymer solutions on the delivery and viability of cells. Three different solutions were used: Hanks Balanced Salt Solution (HBSS) buffer, 0.2 wt-% poly(ethylene oxide) solution (in HBSS), and 0.2 wt-% poly(4-vinyl phenol) (PVP), again in HBSS. The initial mixture of cells (human skeletal myoblasts) and solution was prepared in a 50 mL centrifuge tube. Each solution was then placed in a 5 cc EFD syringe and automatically delivered using an EFD Fluid Dispenser, Model 1500XL (Medtronic Vascular, Santa Rosa, Calif.) at 80 psi (550 kPa). Cells were delivered through a catheter made of polyetheretherketone (PEEK) with a 0.009″ internal diameter and a 30″ length, to microcentrifuge tubes. Data was then collected at T=0 (immediately after delivery) and T=60 minutes (60 minutes after delivery), with readings being carried out in triplicate. Each delivery was approximately 250 μl. To provide data, hemocytometer counts and trypan Blue viability staining were conducted on each solution at the times indicated. Data was also obtained for the HBSS solution before running it through the catheter (the “initial” readings). The results are shown as a bar graph in FIG. 3. The results indicate that cell viability was most stable for cells in the PEO solution, with little change in cell concentration seen between T=0 and T=60, whereas both HBSS and PVP showed a nearly 50% decrease in cell concentration over that time.

An evaluation of the capability of cells from the various tested solutions to proliferate was also conducted. Proliferating human skeletal myoblasts (obtained from CELL SYSTEMS, Inc.), at 70-80% confluency, were harvested by trypsinization (0.25% Trypsin/EDT solution) and counted on a hemacytometer. The cells were then divided into the various test solutions (HBSS, or 0.2% PEO, or 0.2% PVP) so that the final cell density in each solution was approximately one million cells per ml. Tubes containing the cells and the test solutions were left at room temperature for t=0 minutes and t=60 minutes. At time t=0, approximately 250 μl of cell solution was delivered through the catheter tubing under 80 psi (550 kPa) directly into two T-75 tissue culture flasks (VWR). The flasks were supplemented with 10 ml of growth media (M199 basal medium, 10% fetal bovine serum, 1% antibiotic solution; SIGMA CHEMICAL) and placed in the incubator (37° C.) for three days. At time t=60 minutes, the same process was repeated. At time points t=0 and 60 minutes, cell viability was also assessed using trypan blue stain. Damaged or non-viable cells take up the dye and stain blue, while viable cells with intact cell membranes exclude the dye. From the relative amounts of viable and non-viable cell counts a percent viability score can be generated.

On day 3, the tissue culture flasks were removed from the incubator and the attached cells were dissociated and harvested by trypsinization. Prior to removal from the flasks, the cells were observed under a microscope for general gross signs of obvious toxicity. Evaluation of toxicity was based on whether the cells were attached and spread on the surface, which is an indication of general good health, or whether the cells were found floating, an indication of toxicity. The harvested cells were then counted using a hemacytometer. The relative increase (expressed as a “fold”) was determined by dividing the total cell count at t=3 days by the initial seeding count at t=0. The results are shown in Table 1, below:

TABLE 1
3 day proliferation data for Human Skeletal Myoblasts

| | | | | | fold |
|---|---|---|---|---|---|
| | | counts | prolif. | avg (cell number) | increase |
| HBSS (initial) | 1D | 24 | 720000 | 615000 | 2.84 |
| | 1E | 17 | 510000 | | |
| HBSS (t = 0) | 2D | 14 | 420000 | 510000 | 1.89 |
| | 2E | 20 | 600000 | | |
| HBSS (t = 60) | 5D | 12 | 360000 | 360000 | 2.21 |
| | 5E | 12 | 360000 | | |
| 0.2% PEO (t = 0) | 3D | 29 | 870000 | 825000 | 3.43 |
| | 3E | 26 | 780000 | | |
| 0.2% PEO (t = 60) | 6D | 28 | 840000 | 840000 | 3.36 |
| | 6E | 28 | 840000 | | |
| 0.2% PVP (t = 0) | 4E | 28 | 840000 | 690000 | 2.11 |
| | 4E | 18 | 540000 | | |
| 0.2% PVP (t = 60) | 7D | 26 | 520000 | 380000 | 1.94 |
| | 7E | 12 | 240000 | | |

As can be seen from the data in Table 1, the greatest increase in cell proliferation was seen from cells that were placed in 0.2 wt-% PEO solution, indicating that the 0.2% PEO solution is very biocompatible.

### Example 3

### Effect of PEO Polymers on Cell Settling

A set of experiments were conducted to determine the effect of varying molecular weights of 0.2% PEO solution on cell settling over time. First, a variety of 0.2% PEO solutions were prepared in phosphate buffered saline (PBS) solution (pH 7.4) by diluting the corresponding 1.0% solutions. The PEO used was obtained from SIGMA-ALDRICH (Milwaukee, Wis.). The molecular weights of PEO used were 8 million (8M), 1 million (1M), 400 thousand (400K), and 100 thousand (100K) daltons. An additional high concentration (9.5%) PEO solution using PEO with a molecular weight of 8,000 (8K) was also prepared. Human dermal fibroblasts were then prepared and divided equally amongst the different test solutions so that each test solution received approximately 3 million cells in a 0.8 ml of test solution, for a final cell concentration of ˜3.75 million cells/ml.

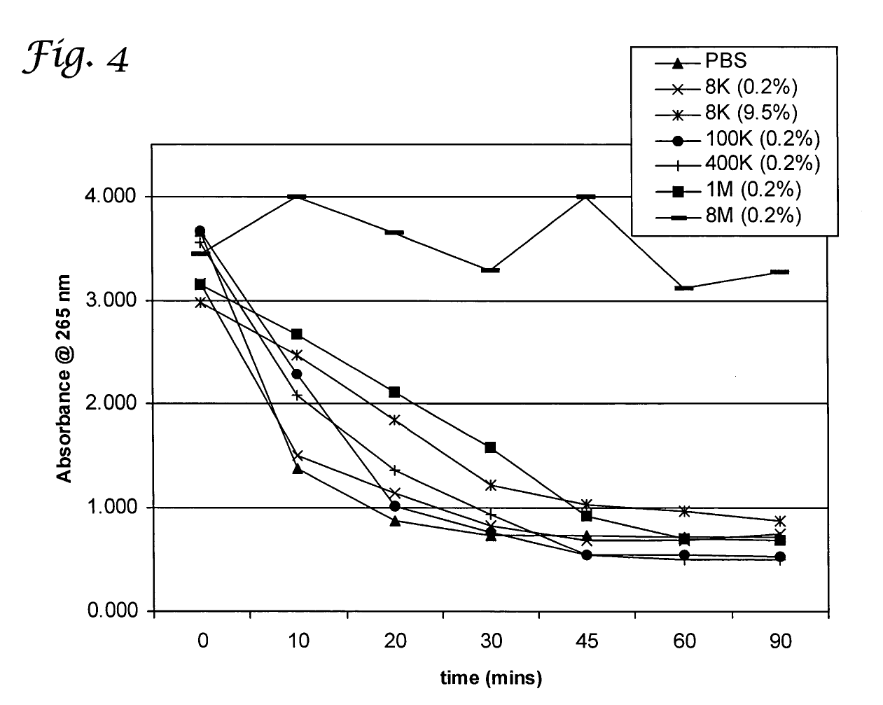

The well-dispersed cell/polymer solutions were then placed into transparent acrylic cuvettes and the turbidity of the solutions was determined in a UV-Vis spectrophotometer. The same cuvettes were read periodically in the spectrophotometer for changes in UV absorption at 0, 10, 20, 30, 45, 60 and 90 minutes. Prior to each reading of the cell/polymer solutions, each condition was blanked using an appropriate cell-free polymer solution. Isotonic isovue 370 solution was used as a positive control. The following data was obtained, which is represented graphically in FIG. 4, shown below in Table 2.

TABLE 2

| | Time | | | | | | |
|---|---|---|---|---|---|---|---|
| Conditions | 0 | 10 | 20 | 30 | 45 | 60 | 90 |
| PBS | 3.671 | 1.378 | 0.876 | 0.730 | 0.734 | 0.723 | 0.719 |
| 8K (0.2%) | 3.173 | 1.502 | 1.140 | 0.835 | 0.694 | 0.691 | 0.750 |
| 8K (9.5%) | 2.984 | 2.474 | 1.836 | 1.218 | 1.035 | 0.966 | 0.875 |
| 100K (0.2%) | 3.678 | 2.276 | 1.023 | 0.764 | 0.552 | 0.552 | 0.526 |
| 400K (0.2%) | 3.569 | 2.074 | 1.355 | 0.932 | 0.550 | 0.498 | 0.496 |
| 1M (0.2%) | 3.161 | 2.665 | 2.117 | 1.583 | 0.924 | 0.700 | 0.689 |
| 8M (0.2%) | 3.452 | 4.000 | 3.660 | 3.302 | 4.000 | 3.122 | 3.274 |

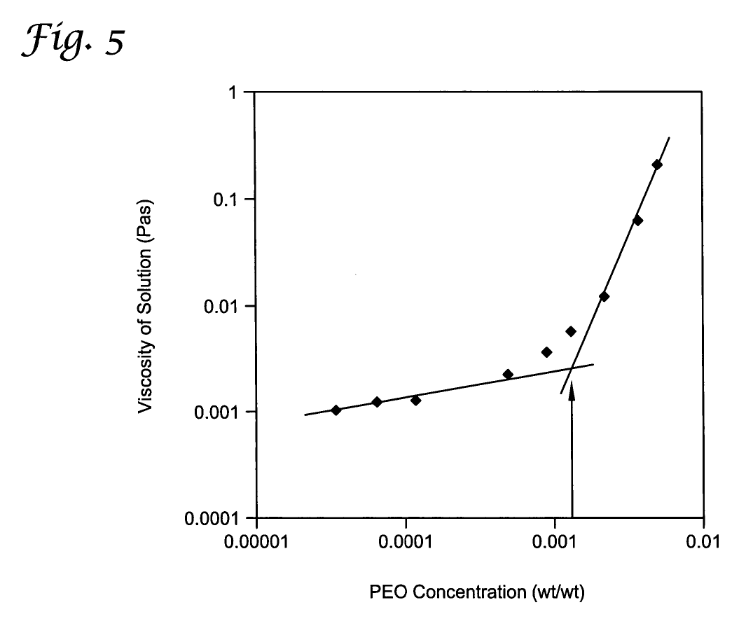

The data obtained indicated that the 8M solution prevented cell settling over the duration of the experiment than the other solutions used (see FIG. 5). The up and down fluctuations observed were most likely due to handling artifacts as cuvettes were moved from one location to the other. Overall cell settling was insignificant with the 8M condition, and compared well with the results obtained with isotonic isovue at 90 minutes. The turbid cuvettes indicate prevention of cell settling while the clear cuvettes indicate settled cells. The cells in a solution of PBS only were substantially settled as early as 10 minutes. The other molecular weight solutions of PEO suspended cells to a lesser extent than the 8M solution. The next best performing solution was the 1M solution, which appeared to significantly slow cell settling.

### Example 4

### Deliverability of Various PEO Solutions

A set of experiments were conducted to determine the “deliverability” of varying molecular weights and concentrations of PEO solutions through IntraLume catheters; and to measure flow rates for manual and automated delivery. First, a variety of solutions with varying concentrations of PEO (SIGMA-ALDRICH, Milwaukee, Wis.) in phosphate buffered saline (PBS) (pH 7.4) were prepared. The following solutions were prepared:

1. 8M (MW=8 million), 1 wt-% PEO solution

2. 1M (MW=1 million), 1 wt-% PEO solution

3. 400K (MW=400 thousand), 2 wt-% PEO solution

4. 100K (MW=100 thousand), 2 wt-% PEO solution

5. 8M (MW=8 million), 0.2 wt-% PEO solution

6. 1M (MW=1 million), 0.2 wt-% PEO solution

7. 400K (MW=400 thousand), 0.2 wt-% PEO solution

8. 100 K (MW=100 thousand), 0.2 wt-% PEO solution

The solutions were then manually delivered using MICROLUME SL infusion catheters (Medtronic Vascular, Part #DH 12290). The catheter used had a length of 177.5 cm, a proximal (hub) ID of 0.0085″, and a distal (tip) ID of 0.007″. To conduct manual delivery, a 1 cubic centimeter (cc) syringe was loaded with the PEO solution of interest. The syringe (1 cc LuerLok) was then attached to the hub of the infusion catheter, and the solution was delivered through the catheter by manually advancing the plunger of the syringe. A stopwatch was used to obtain the delivery time for a specific volume. By recording the time and volume of delivery, the flow rate for each PEO solution could then be calculated.

Solutions were also delivered by automated delivery using an EFD Compressed Air Powered Fluid Dispenser with 3 cc syringe barrels and pistons. A 3 cc syringe was secured to the catheter, filled with the PEO solution to be tested, capped with a piston, and then attached to the dispenser. The dispenser pressure was set to 80 psi (550 kPa), and the PEO solution was then delivered into a tared microcentrifuge tube, recording the weight delivered. The flow rate for the PEO solution was then calculated.

The results and observations for each PEO solution are shown in Table 3 below:

TABLE 3

| | | Flow rate (uL/sec) | Flow rate (uL/sec) | |
|---|---|---|---|---|
| | | Hand delivery | Automated delivery | |
| PEO MW | wt-% solution | (approx. 160 psi) | (80 psi) | Comments |
| 8M | 1.0% | 2.8 | 0 | Automated delivery impossible; |
| | | | | manual delivery very difficult |
| 1M | 1.0% | 5.9 | 1.7 | Manual delivery difficult |
| 400K | 2.0% | 2.1 | 1.1 | Manual delivery almost impossible |
| 100K | 2.0% | 15.0 | 3.7 | Manual delivery reasonable |
| 8M | 0.2% | 14.3 | 4.2 | Manual delivery reasonable |
| 1M | 0.2% | 23.6 | 7.8 | Manual delivery reasonable |
| 400K | 0.2% | 40.2 | 10.4 | Manual delivery reasonable |
| 100K | 0.2% | 46.2 | 14.0 | Manual delivery reasonable |

As can be seen from Table 3, all of the 0.2 wt-% PEO solutions were deliverable through the MICROLUME catheter, with flow rates increasing with increasing delivery pressure and with decreasing molecular weight. The 1 wt-% and 2 wt-% PEO solutions were more difficult to deliver than their 0.2 wt-% counterparts. The 8M MW (1.0 wt-%) solution was undeliverable at 80 psi (550 kPa) delivery pressure, and all of the high concentration (1-2 wt-%) solutions were difficult to deliver, except for the 2.0 wt-% solution at 100K MW.

### Example 5

### Viscosity of PEO Solutions

The viscosity of various PEO solutions was evaluated using various techniques. First, the viscosity of an 8,000,000 dalton (8M) MW PEO solution in phosphate buffered saline (pH 7.4) was evaluated. The PEO was obtained from SIGMA-ALDRICH (Milwaukee, Wis.). The viscosity was measured with an Ubbelohde-type capillary viscometer at room temperature. The results are shown in FIG. 5. The graph shows an “overlap” transition at 0.13 wt-%. The overlap transition, as discussed herein, represents the point where individual coil chains start to overlap in solution, resulting in a change in viscosity due to coil entanglement. FIG. 5 illustrates how the viscosity of the 8M solution of PEO increased hundreds of times after adding just 0.5 wt-% of PEO.

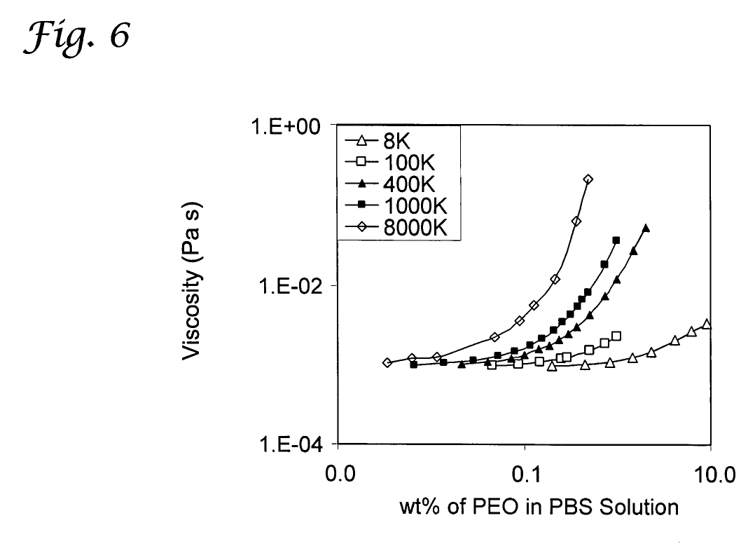

The effects of various molecular weights of PEO on the rheological properties of PEO solutions were then evaluated. Five PEOs with varying molecular weights were used. The MW (g/mol) and SIGMA-ALDRICH catalog number for the polymers were: 8,000 (SA#20245-2), 100,000 (SA#18198-6), 400,000 (SA#37277-3), 1,000,000 (SA#372781), and 8,000,000 (SA#372838). The PEO samples were again prepared by dissolving the PEO polymers in PBS (pH 7.4). The concentrations of the solutions were varied from 0.005 wt-% to 10 wt-% for each of the solutions, and the viscosity of the solutions was measured with an Ubbelohde-type capillary viscometer at room temperature. Water, with a viscosity of about 0.00 Pa s (Pascal-second), was used as a reference. The results are shown in FIG. 6. The viscosity of the 8M molecular weight solution was higher than that of the 8,000 molecular weight PEO by about 10 times at the concentration of 0.2 wt-%, and about 100 times higher at 0.5 wt-%.

The steady shear viscosity versus shear rate of an 8M PEO solution in phosphate buffered saline (pH 7.4) was also evaluated. The measurements were conducted at room temperature with 50 mm diameter parallel disks mounted to an ARES (Advanced Rheometric Expansion System) strain control viscometer (TA INSTRUMENTS, Piscataway, N.J.), which is capable of subjecting a sample to either a dynamic (sinusoidal) or steady (linear) shear strain (deformation), then measuring the resultant torque expended by the sample in response to the strain. The results are shown in FIG. 7. The results demonstrated that the viscosity of the 0.5 wt-% solution dropped by almost two orders of magnitude when the shear rate increased from 0.1 to 1000 s⁻¹. A reduction of about one order of magnitude was observed in the solution of 0.2 wt-% when the shear rate was increased from 1 to 1000 s⁻¹. Shear thinning was not significant in solutions of 0.05 wt-% and 0.01 wt-%, as these concentrations were lower than the “overlap” concentration.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A cell delivery composition, comprising:
a biocompatible carrier liquid;
a biocompatible shear thinning polymer at a concentration from greater than or equal to the shear thinning polymer's overlap concentration in the biocompatible carrier liquid up to 10 wt-% concentration of the shear thinning polymer in the biocompatible carrier liquid; and
a plurality of cells.

2. The composition of claim 1, wherein the shear thinning polymer has a molecular weight of 1,000,000 g/mol or more.

3. The composition of claim 1, wherein the shear thinning polymer is present at a concentration of 2 wt-% or less in the biocompatible carrier liquid.

4. The composition of claim 1, wherein the shear thinning polymer is a poly(alkylene oxide) polymer.

5. The composition of claim 4, wherein the poly(alkylene oxide) polymer is selected from the group consisting of poly(ethylene oxide), poly(propylene oxide), and poly(ethylene-co-propylene oxide) copolymers, and combinations thereof.

6. The composition of claim 5, wherein the shear thinning polymer is poly(ethylene oxide).

7. The composition of claim 6, wherein the poly(ethylene oxide) is present at a concentration of 0.1 wt-% to 2.0 wt-% in the biocompatible carrier liquid.

8. The composition of claim 5, wherein the poly(ethylene oxide) has a molecular weight of 1,000,000 g/mol or more.

9. The composition of claim 8, wherein the poly(ethylene oxide) has a molecular weight of 8,000,000 g/mol or more.

10. The composition of claim 1, wherein the cells are selected from the group consisting of islet cells, stem cells, hepatocytes, chondrocytes, osteoblasts, neuronal cells, glial cells, smooth muscle cells, endothelial cells, nucleus pulposus cells, epithelial cells, myoblasts, myocytes, macrophages, purkinje cells, erythrocytes, platelets, fibroblasts, and combinations thereof.

11. The composition of claim 1, wherein the cells are suitable for the regeneration of cardiac tissue.

12. The composition of claim 1, wherein the cells have a settling rate of 1 millimeter per hour or less in the cell delivery composition when it is not subjected to shear stress.

13. The composition of claim 1, wherein the cell delivery composition exhibits a one order magnitude decrease in viscosity when the shear rate is increased from 1 s⁻¹ to 1000 s⁻¹.

14. The composition of claim 1, further comprising a polypeptide.

15. The composition of claim 14, wherein the polypeptide is a buffering protein or growth factor.

16. The composition of claim 14, wherein the polypeptide is selected from the group consisting of PDGF, VEGF, FGF, EGF, IGF, TGF-beta, MGF, cytokines, prostaglandins, collagens, elastin, fibronectin, laminin, tenascin, entactin, fibrinogen, fibrin, heparin, heparin sulfate, dermatan sulfate, keratin sulfate, and chondroitin sulfate.

17. A method of delivering cells to a subject, comprising:
providing a cell delivery composition comprising a biocompatible carrier liquid;
a biocompatible shear thinning polymer at a concentration from greater than or equal to the shear thinning polymer's overlap concentration in the biocompatible carrier liquid up to 10 wt-% concentration of the shear thinning polymer in the biocompatible carrier liquid; and a plurality of cells, and
delivering the cell delivery composition to a tissue site in the subject.

18. The method of claim 17, wherein the shear thinning polymer has a molecular weight of 1,000,000 g/mol or more and is present at a concentration of 2 wt-% or less in the biocompatible carrier liquid.

19. The method of claim 17, wherein the shear thinning polymer is a poly(alkylene oxide) polymer.

20. The method of claim 19, wherein the shear thinning polymer is a poly(ethylene oxide).

21. The method of claim 21, wherein the poly(ethylene oxide) is present at a concentration of 0.1 wt-% to 2.0 wt-% in the biocompatible carrier liquid.

22. The method of claim 21, wherein the poly(ethylene oxide) has a molecular weight of 1,000,000 g/mol or more.

23. The method of claim 17, wherein the cells have a settling rate of 1 millimeter per hour or less in the cell delivery composition when it is not subjected to shear stress.

24. The method of claim 17, wherein the cell delivery composition exhibits a one order magnitude decrease in viscosity when the shear rate is increased from 1 s⁻¹ to 1000 s⁻¹.

25. The method of claim 17, wherein the cells are present at a concentration from 1×10⁶ cells per milliliter to 1×10⁹ cells per milliliter in the cell delivery composition.

26. The method of claim 17, wherein the tissue site comprises cardiac tissue.

27. The method of claim 17, wherein 70% or more of the cells remain viable after delivery to the tissue site.

28. The method of claim 27, wherein the cells are retained at the tissue site for at least 24 hours.

29. A method of cardiovascular regeneration, comprising:
providing a cell delivery composition, comprising
a biocompatible carrier liquid;
a poly(ethylene oxide) polymer with a molecular weight of 1,000,000 g/mol or more at a concentration of 0.1 wt-% to 2.0 wt-% in the biocompatible carrier liquid; and
a plurality of mammalian cells suitable for cardiovascular application, and
delivering the cell delivery composition including the mammalian cells at a constant rate to a cardiac tissue site in a mammal, wherein 70% or more of the mammalian cells remain viable after delivery to the cardiac tissue site.
